(19) United States
(12) Patent Application Publication
(10) Pub. No.: US 2003/0220517 A1
(43) Pub. Date: Nov. 27, 2003

(54) Method for producing ketocarboxylic acid derivatives

(76) Inventor: Albrecht Marhold, Leverkusen (DE)

Correspondence Address: BAYER CROPSCIENCE LP, 100 BAYER ROAD, PITTSBURGH, PA, 15205, US

(21) Appl. No.: 10/380,439

(22) PCT Filed: Sep. 10, 2001
(86) PCT No.: PCT/EP01/10432

(30) Foreign Application Priority Data
Sep. 22, 2000 (DE) 100 47 118.8

(51) Int. Cl. C07C 317/24; C07C 253/30; C07C 231/12; C07C 51/373

(52) U.S. Cl. 558/359; 560/11; 560/20; 560/41; 560/51; 562/459; 562/450; 562/429; 562/434

(57) ABSTRACT

A novel process affords ω-ketocarboxylic acid derivatives of the formula (I)

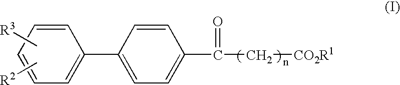

in which

R¹, R², R³, and n are as defined in the description

by reaction of compounds of the formula (II)

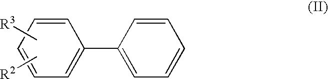

with compounds of the formula (III)

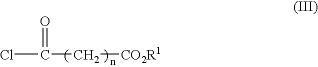

or with compounds of the formula (IV)

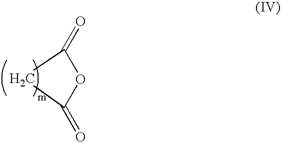

in which

m is as defined in the description

in anhydrous hydrogen fluoride, if appropriate in the presence of boron trifluoride.

Novel ω-ketocarboxylic acid derivatives of the formula (If)

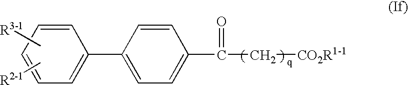

in which

R¹⁻¹, R²⁻¹, R³⁻¹ and q are as defined in the description.

The present invention relates to a novel process for preparing ω-ketocarboxylic acid derivatives, and to their use as intermediates for the synthesis of insecticidally active cyclic imines.

It is known that biphenylketocarboxylic acids can be prepared by reacting unsubstituted biphenyl with acid anhydrides (cf. Org. Prep. Proc. Int. 1995, 27, 550-552) or with ketocarbonyl chlorides (cf. J. C. S. Perkin Trans 2 1983, 1455-1461) in the presence of aluminium chloride.

However, these methods can only be used if the starting material employed is unsubstituted biphenyl, or if the substituents do not react with aluminium chloride. It is not possible to prepare biphenyls carrying fluorinated side-chains in this manner, since the catalyst reacts with the side-chain first, resulting in partial or even complete exchange of the fluorine atoms for chlorine. Because of this, the known methyl 4-(4′-trifluoromethylbiphenyl)-4-oxobutyrate is, as described in WO 98/09940 prepared by Suzuki coupling from 4-trifluoromethylphenylboronic acid and methyl 4-(4-bromophenyl)-4-oxobutyrate.

The present invention relates to a novel process for preparing ω-ketocarboxylic acid derivatives of the formula (I)

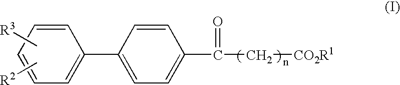

in which

R¹ represents hydrogen or straight-chain or branched alkyl,

R² represents halogen or in each case fluorine-substituted, in each case straight-chain or branched alkyl, alkoxy or alkylthio,

R³ represents hydrogen, halogen, in each case fluorine-substituted, in each case straight-chain or branched alkyl alkoxy or alkylthio; represents in each case straight-chain or branched alkyl, alkoxy, alkylthio; cyano, nitro, —CO₂R⁴, —CONR⁵R⁶ or —SO₂R⁷,

R² and R³ furthermore together represent —O-(halo)alkyl-O—, if the two radicals are located ortho to one another,

R⁴ represents optionally fluorine-substituted straight-chain or branched alkyl,

R⁵ and R⁶ independently of one another represent hydrogen or straight-chain or branched alkyl,

R⁷ represents halogen or represents optionally fluorine-substituted straight-chain or branched alkyl,

n represents 1, 2, 3 or 4,

characterized in that compounds of the formula (II)

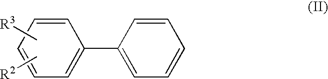

in which R² and R³ are as defined above are reacted either

a) with compounds of the formula (III)

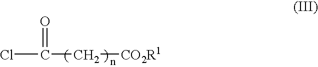

in which R¹ and n are as defined above or

b) with compounds of the formula (IV)

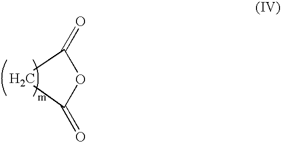

in which

m represents 2 or 3,

in anhydrous hydrogen fluoride, if appropriate in the presence of boron trifluoride.

It is extremely surprising that ω-ketocarboxylic acid derivatives of the formula (I) can be prepared by the process according to the invention in a smooth reaction without interfering side reactions. Thus, according to the present prior art, it would have been expected that, in the case of biphenyls carrying fluorinated side-chains, under the conditions of a Friedel-Crafts acylation there would initially be a reaction of the catalyst with the side-chain. Under the reaction conditions of the reaction according to the invention, such an unwanted reaction is avoided. Moreover, the process according to the invention affords the co-ketocarboxylic acid derivatives of the formula (I) after a short reaction time in pure form, with surprising selectivity and in good yields.

The process according to the invention has a number of advantages. Thus, compared to a synthesis of the biphenyl skeleton by Suzuki coupling, the synthesis of the ω-ketocarboxylic acid derivatives of the formula (I) can be realized industrially in a very simple manner. It is furthermore favourable that the catalyst can be recovered by distillation, which renders the process very environmentally friendly.

Using, for example, 4-trifluoromethoxybiphenyl and methyl 4-oxo-4-chlorobutyrate as starting materials and hydrogen fluoride/boron trifluoride as catalyst, the course of the process according to the invention, variant (a), can be represented by the formula scheme below:

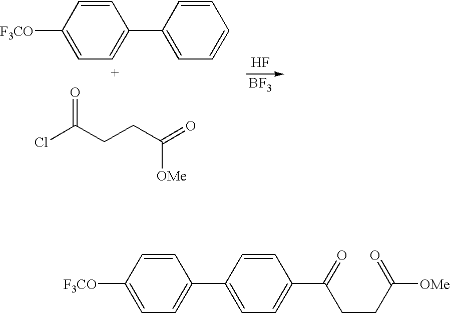

Using, for example, 4-trifluoromethoxybiphenyl and succinic anhydride as starting materials and hydrogen fluoride/boron trifluoride as catalyst, the course of the process according to the invention, variant (b), can be represented by the following formula scheme:

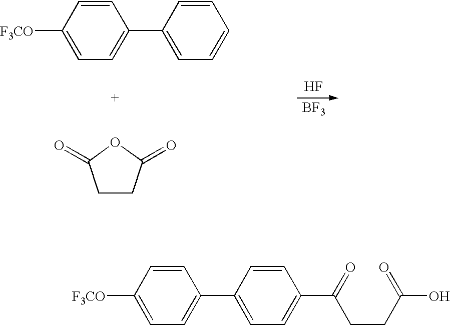

The formulae (II), (III) and (IV) provide general definitions of the compounds required as starting materials for the process according to the invention.

Preferred substituents or ranges of the radicals in the formnulae, mentioned above and below, of starting materials of the formula (II) are illustrated below.

R² preferably represents fluorine, chlorine, bromine, iodine or represents in each case straight-chain or branched C₁-C₄-alkyl, C₁-C₄-alkoxy or C₁-C₄-alkylthio, each of which is substituted by 1 to 9 fluorine atoms.

R³ preferably represents hydrogen, fluorinie, chlorine, bromine, represents in each case straight-chain or branched C₁-C₄-alkyl, C₁-C₄-alkoxy, C₁-C₄-alkylthio, each of which is substituted by 1 to 9 fluorine atoms; represents in each case straight-chain or branched C₁-C₄-alkyl, C₁-C₄-alkoxy, C₁-C₄-alkylthio; cyano, nitro, —CO₂R⁴, —CONH₂ or —SO₂R⁷.

R² and R³ furthermore together preferably represent —O—(C₁-C₄-alkyl)-O— if the two radicals are located ortho to one another, where the alkyl moiety may optionally be substituted by 1 to 8 fluorine atoms.

R⁴ preferably represents straight-chain or branched C₁-C₄-alkyl which is optionally substituted by 1 to 9 fluorine atoms.

R⁷ preferably represents fluorine, chlorine or represents straight-chain or branched C₁-C₄-alkyl which is optionally substituted by 1 to 9 fluorine atoms.

R² particularly preferably represents fluorine, chlorine, bromine or represents methyl, ethyl, methoxy, ethoxy, methylthio or ethylthio, each of which is substituted by 1 to 5 fluorine atoms.

R³ particularly preferably represents hydrogen, fluorine, chlorine, represents methyl, ethyl, methoxy, ethoxy, methylthio or ethylthio, each of which is substituted by 1 to 5 fluorine atoms; represents methyl, ethyl, methoxy, ethoxy, nitro, —CO₂R⁴ or —SO₂R⁷,

R² and R³ furthermore together particularly preferably represent —O—CH₂—O— or —O—CH₂—CH₂—O— if the two radicals are located ortho to one another, where the methylene or ethylene moiety may optionally be substituted by 1 to 4 fluorine atoms.

R⁴ particularly preferably represents methyl or ethyl, optionally substituted by 1 to 5 fluorine atoms.

R⁷ particularly preferably represents fluorine, chlorine or represents methyl or ethyl, optionally substituted by 1 to 5 fluorine atoms.

R² very particularly preferably represents fluorine, chlorine, bromine or represents —CF₃, —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H.

R³ very particularly preferably represents hydrogen, fluorine, chlorine, methyl, ethyl, methoxy, ethoxy, —CF₃, —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H, nitro, —CO₂R⁴ or —SO₂R⁷.

R² and R³ furthermore together very particularly preferably represent —O—CH₂—O—, —O—CH₂—CH₂—O—, —O—CF₂—O—, —O—CF₂—CF₂—O—, if the two radicals are located ortho to one another.

R⁴ very particularly preferably represents methyl, ethyl, —CF₃, —CF₂CF₃ or —CF₂H.

R⁷ very particularly preferably represents fluorine, methyl, ethyl, —CF₃, —CF₂CF₃ or —CF₂H.

Preferred substituents or ranges of the radicals in the formulae, mentioned above and below, of starting materials of the formula (III) are illustrated below.

R¹ preferably represents hydrogen or straight-chain or branched C₁-C₄-alkyl.

n preferably represents 2, 3 or 4.

R¹ particularly preferably represents hydrogen, methyl, ethyl, n-propyl or isopropyl.

n particularly preferably represents 2, 3 or 4.

R¹ Very particularly preferably represents hydrogen, methyl or ethyl.

n very particularly preferably represents 2.

Preferred substituents or ranges of the radicals in the formulae, mentioned above and below, of starting materials of the formula (IV) are illustrated below.

m preferably represents 2 or 3.

m particularly preferably represents 2 or 3.

m very particularly preferably represents 2.

Starting materials for the process according to the invention which are particularly emphasized are compounds of the formula (IIa)

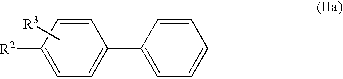

in which

R² and R³ are as defined above.

Moreover, starting materials for the process according to the invention which are particularly emphasized are compounds of the formula (IIb)

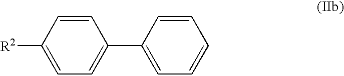

in which

R² is as defined above.

Starting materials for the process according to the invention which are particularly emphasized are compounds of the formula (IIIa)

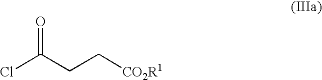

in which

R¹ is as defined above.

A starting material for the process according to the invention which is particularly emphasized is the compound of the formula (IVa)

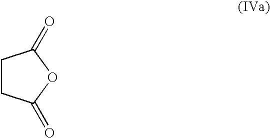

If compounds of the formulae (IIa) and (IIIa) are used, the end products of the process according to the invention obtained are compounds of the formula (Ia)

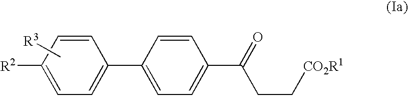

If compounds of the formnulae (IIb) and (IIIa) are used, the end products of the process according to the invention obtained are compounds of the formula (Ib)

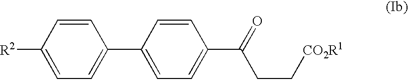

If compounds of the formulae (IIa) and (IVa) are used, the end products of the process according to the invention obtained are compounds of the formula (Ic)

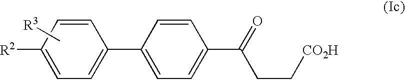

If compounds of the formulae (IIb) and (IVa) are used, the end products of the process according to the invention obtained are compounds of the formula (Id)

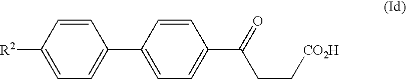

The abovementioned general or preferred radical definitions or illustrations can be combined with one another as desired, i.e. including combinations between the respective ranges and preferred ranges. The definitions apply both to the end products and, correspondingly, to precursors and intermediates.

Saturated hydrocarbon radicals, such as alkyl, can in each case be straight-chain or branched as far as this is possible, including in combination with heteroatoms, such as, for example, in alkoxy; C₄-alkyl, for example, is meant to include both n-butyl and t-butyl.

Optionally substituted radicals can be mono- or polysubstituted.

Preferred for use in the process according to the invention are those compounds of the formulae (II), (III) and (IV) which contain a combination of the meanings listed above as being preferred.

Particularly preferred for use in the process according to the invention are those compounds of the formulae (II), (III) and (IV) which contain a combination of the meanings listed above as being particularly preferred.

Very particularly preferred for use in the process according to the invention are those compounds of the formulae (II), (III) and (IV) which contain a combination of the meanings listed above as being very particularly preferred.

The starting materials of the formula (II) are known or can be prepared by known processes.

The starting materials of the formulae (III) and (IV) are known.

When carrying out the process according to the invention, variants (a) and (b) are generally each carried out under elevated pressure, preferably at a pressure between 2 bar and 20 bar, particularly preferably between 3 bar and 10 bar, very particularly preferably between 3 bar and 6 bar.

The reaction temperatures for carrying out the process according to the invention, variants (a) and (b), can in each case be varied within a relatively wide range. In general, the process is carried out at temperatures between −20° C. and +80° C., preferably between −10° C. and +40° C.

The compounds of the formulae (III) and (IV), respectively, are generally employed in a molar ratio of from 0.5:1 to 10:1, preferably from 1:1 bis 5:1, particularly preferably of 1.5:1, based on the starting material of the formula (II). However, it is also possible to choose other ratios of the reaction components.

In general, the reaction is carried out by metering the starting material of the formula (II) and the compound of the formula (III) or (IV) into an initial charge of hydrogen fluoride, followed, if appropriate, by the addition of boron trifluoride, and the reaction is carried out at the desired temperature and the desired pressure.

For work-up, in general hydrogen fluoride/boron trifluoride is removed by distillation at the desired temperature and the residue is added to ice and extracted with an organic solvent. The product can be freed from any impurities by recrystallization.

Some of the ω-ketocarboxylic acid derivatives of the formula (I) formed in the process according to the invention are known. Their use as inhibitors of metallo-proteinases has also been described (cf. WO 98/09940).

Moreover, the ω-ketocarboxylic acid derivatives of the formula (I) which can be prepared according to the invention are useful intermediates in the preparation of active compounds for controlling pests. The compounds are particularly suitable for preparing cyclic imines for controlling insects, arachnids and nematodes encountered in agriculture, in forests, in the protection of stored products and the protection of materials and also in the hygiene sector (see WO 98/22438).

Thus, cyclic imines of the formula (V)

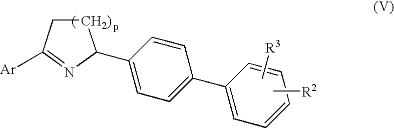

in which

R² and R³ are as defined above

Ar represents mono- to trisubstituted phenyl and

p represents 1, 2 or 3,

can be prepared, for example,

by reacting ω-ketocarboxylic acid derivatives of the formula (Ie)

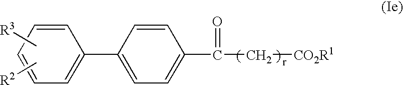

in which R¹, R² and R³ are as defined above and

r represents 2, 3 or 4,

in a first step with 2-amino-2-phenylethanol in the presence of an acid (for example toluenesulphonic acid) and a diluent (for example toluene) and reacting the resulting intermediates of the formula (VI)

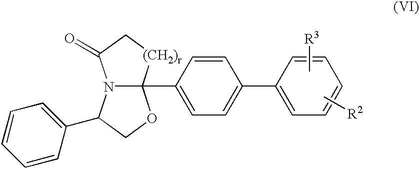

in which R², R³ and r are as defined above

in a second step in the presence of a Lewis acid (for example TiCl₄), in the presence of a reducing agent (for example Et₃SiH) and in the presence of a diluent (for example dichloromethane) and reacting the resulting intermediates of the formula (VII)

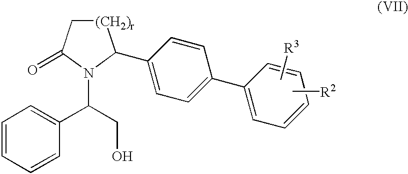

in which R², R³ and r are as defined above

in a third step with a chlorinating agent (for example thionyl chloride) in the presence of a diluent (for example THF) and reacting the resulting intermediates of the formula (VIII)

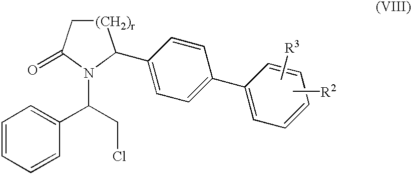

in which R², R³ and r are as defined above

in a fourth step with a base (for example KO^{t}Bu) in the presence of a diluent (for example tert-butanol) and reacting the resulting intermediates of the formula (IX)

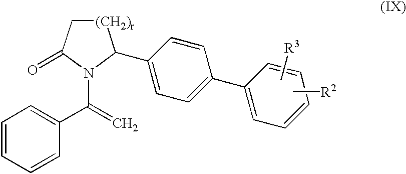

in which R², R³ and r are as defined above

in a fifth step with an acid (for example HCl) in the presence of a diluent (for example THF) and reacting the resulting intermediates of the formula (X)

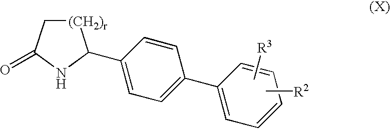

in which R², R³ and r are as defined above

in a sixth step with di-tert-butyl dicarbonate in the presence of a base (for example dimethylaminopyridine) and in the presence of a diluent (for example dichloromethane) and reacting the resulting intermediate of the formula (XI)

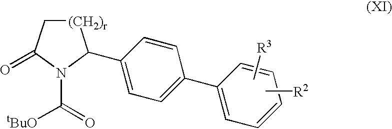

in which R², R³ and r are as defined above

in a seventh step with metallated aromatic compounds of the formula (XII)

Ar—M (XII)

in which Ar is as defined above and

M represents Li, MgCl, MgBr, MgI or ZnCl,

in the presence of a diluent (for example THF) and reacting the resulting intermediates of the formula (XIII)

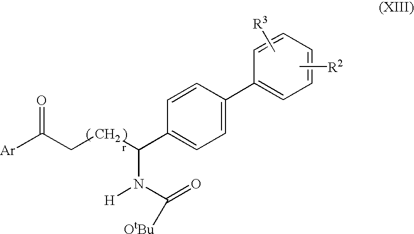

in which Ar, R², R³ and r are as defined above

in an eighth step without prior isolation with an acid (for example trifluoroacetic acid).

The compounds of the formulae (VI), (VII), (VIII), (IX), (X), (XI), (XII) and (XIII) can be prepared by known processes.

The compounds of the formula (If)

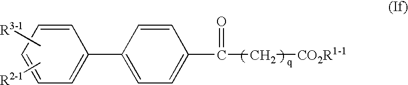

in which

a) R¹⁻¹ represents hydrogen or straight-chain or branched alkyl,

R²⁻¹ represents halogen or in each case fluorine-substituted, in each case straight-chain or branched alkyl, alkoxy or alkylthio,

R³⁻¹ represents hydrogen, halogen, represents in each case fluorine-substituted, in each case straight-chain or branched alkyl, alkoxy or alkylthio; represents in each case straight-chain or branched alky, alkoxy, alkylthio; cyano, nitro, —CO₂R⁴⁻¹, —CONR⁵⁻¹R⁶⁻¹ or —SO₂R⁷⁻¹,

R²⁻¹ and R³⁻¹ furthermore together represent —O-(halo)alkyl-O— if the two radicals are located ortho to one another,

R⁴⁻¹ represents optionally fluorine-substituted straight-chain or branched alkyl,

R⁵⁻¹ and R⁶⁻¹ independently of one another represent hydrogen or straight-chain or branched alkyl,

R⁷⁻¹ represents halogen or represents optionally fluorine-substituted straight-chain or branched alky,

q represents 1, 3 or 4, or

b) R¹⁻¹ represents hydrogen or straight-chain or branched alkyl,

R²⁻¹ represents in each case fluorine-substituted, in each case straight-chain or branched C₂-C₆-alkyl, alkoxy or alkylthio,

R³⁻¹ represents hydrogen, halogen, represents in each case fluorine-substituted, in each case straight-chain or branched alkyl, alkoxy or alkylthio; represents in each case straight-chain or branched alkyl, alkoxy, alkylthio; cyano, nitro, —CO₂R⁴⁻¹, —CONR⁵⁻¹R⁶⁻¹ or —SO₂R⁷⁻¹,

R²⁻¹ and R³⁻¹ furthermore together represent —O-(halo)alkyl-O— if the two radicals are located ortho to one another,

R⁴⁻¹ represents optionally fluorine-substituted straight-chain or branched alkyl,

R⁵⁻¹ and R⁶⁻¹ independently of one another represent hydrogen or straight-chain or branched alkyl,

R⁷⁻¹ represents halogen or represents optionally fluorine-substituted straight-chain or branched alkyl,

q represents 2

are novel and also form part of the subject-matter of this invention.

Preference is given to compounds of the formula (If) in which

a) R¹⁻¹ represents hydrogen or straight-chain or branched C₁-C₄-alkyl,

R²⁻¹ represents fluorine, chlorine, bromine, iodine or represents in each case straigbt-chain or branched C₁-C₄-alkyl, C₁-C₄-alkoxy or C₁-C₄-alkylthio, each of which is substituted by 1 to 9 fluorine atoms,

R³⁻¹ represents hydrogen, fluorine, chlorine, bromine, represents in each case straight-chain or branched C₁-C₄-alkyl, C₁-C₄-alkoxy, C₁-C₄-alkylthio, each of which is substituted by 1 to 9 fluorine atoms; represents in each case straight-chain or branched C₁-C₄-alkyl, C₁-C₄-alkoxy, C₁-C₄-alkylthio; cyano, nitro, —CO₂R⁴⁻¹, —CONH₂ or —SO₂R⁷⁻¹,

R²⁻¹ and R³⁻¹ furthermore together represent —O—(C₁-C₄-alkyl)-O— if the two radicals are located ortho to one another, where the alkyl moiety may optionally be substituted by 1 to 8 fluorine atoms,

R⁴⁻¹ represents straight-chain or branched C₁-C₄-alkyl which is optionally substituted by 1 to 9 fluorine atoms,

R⁷⁻¹ represents fluorine, chlorine or represents straight-chain or branched C₁-C₄-alkyl which is optionally substituted by 1 to 9 fluorine atoms,

q represents 3 or 4, or

b) R¹⁻¹ represents hydrogen or straight-chain or branched C₁-C₄-alkyl,

R²⁻¹ represents in each case straight-chain or branched C₂-C₄-alkyl, C₁-C₄-alkoxy or C₁-C₄-alkylthio, each of which is substituted by 1 to 9 fluorine atoms,

R³⁻¹ represents hydrogen, fluorine, chlorine, bromine, represents in each to case straight-chain or branched C₁-C₄-alkyl, C₁-C₄-alkoxy, C₁-C₄-alkylthio, each of which is substituted by 1 to 9 fluorine atoms; represents in each case straight-chain or branched C₁-C₄-alkyl, C₁-C₄-alkoxy, C₁-C₄-alkylthio; cyano, nitro, —CO₂R⁴⁻¹, —CONH₂ or —SO₂R⁷⁻¹,

R²⁻¹ and R³⁻¹ together represent —O—(C₁-C₄-alkyl)—O— if the two radicals are located ortho to one another, where the alkyl moiety may optionally be substituted by 1 to 8 fluorine atoms,

R⁴⁻¹ represents straight-chain or branched C₁-C₄-alkyl which is optionally substituted by 1 to 9 fluorine atoms,

R⁷⁻¹ represents fluorine, chlorine or represents straight-chain or branched C₁-C₄-alkyl which is optionally substituted by 1 to 9 fluorine atoms,

q represents 2.

Particular preference is given to compounds of the formula (If) in which

a) R¹⁻¹ represents hydrogen, methyl, ethyl, n-propyl or isopropyl,

R²⁻¹ represents fluorine, chlorine, bromine or represents methyl, ethyl, methoxy, ethoxy, methylthio or ethylthio, each of which is substituted by 1 to 5 fluorine atoms,

R³⁻¹ represents hydrogen, fluorine, chlorine, represents methyl, ethyl, methoxy, ethoxy, methylthio or ethylthio, each of which is substituted by 1 to 5 fluorine atoms; represents methyl, ethyl, methoxy, ethoxy, nitro, —CO₂R⁴⁻¹ or —SO₂R⁷⁻¹,

R²⁻¹ and R³⁻¹ furthermore together represent —O—CH₂—O— or —O—CH₂—CH₂—O— if the two radicals are located ortho to one another, where the methylene or ethylene moiety may optionally be substituted by 1 to 4 fluorine atoms,

R⁴⁻¹ represents methyl or ethyl, optionally substituted by 1 to 5 fluorine atoms,

R⁷⁻¹ represents fluorine, chlorine or represents methyl or ethyl, optionally substituted by 1 to 5 fluorine atoms,

q represents 3 or 4, or

b) R¹⁻¹ represents hydrogen, methyl, ethyl, n-propyl or isopropyl,

R²⁻¹ represents ethyl, methoxy, ethoxy, methylthio or ethylthio, each of which is substituted by 1 to 5 fluorine atoms,

R³⁻¹ represents hydrogen, fluorine, chlorine, represents methyl, ethyl, methoxy, ethoxy, methylthio or ethylthio, each of which is substituted by 1 to 5 fluorine atoms; represents methyl, ethyl, methoxy, ethoxy, nitro, —CO₂R⁴⁻¹ or —SO₂R⁷⁻¹,

R²⁻¹ and R³⁻¹ furthermore together represent —O—CH₂—O— or —O—CH₂—CH₂—O— if the two radicals are located ortho to one another, where the methylene or ethylene moiety may optionally be substituted by 1 to 4 fluorine atoms,

R⁴⁻¹ represents methyl or ethyl, optionally substituted by 1 to 5 fluorine atoms,

R⁷⁻¹ represents fluorine, chlorine or represents methyl or ethyl, optionally substituted by 1 to 5 fluorine atoms,

q represents 2.

Very particular preference is given to compounds of the formula (If) in which

a) R¹⁻¹ represents hydrogen, methyl or ethyl,

R²⁻¹ represents fluorine, chlorine, bromine or represents —CF₃, —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H,

R³⁻¹ represents hydrogen, fluorine, chlorine, methyl, ethyl, methoxy, ethoxy, —CF₃, —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H, nitro, —CO₂R⁴⁻¹ or —SO₂R⁷⁻¹,

R²⁻¹ and R³⁻¹ furthermore together represent —O—CH₂—O—, —O—CH₂—CH₂—O—, —O—CF₂—O—, —O—CF₂—CF₂—O— if the two radicals are located ortho to one another,

R⁴⁻¹ represents methyl, ethyl, —CF₃, —CF₂CF₃ or —CF₂H,

R⁷⁻¹ represents fluorine, methyl, ethyl, —CF₃, —CF₂CF₃ or —CF₂H,

q represents 3 or 4, or

b) R¹⁻¹ represents hydrogen, methyl or ethyl,

R²⁻¹ represents —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H,

R³⁻¹ represents hydrogen, fluorine, chlorine, methyl, ethyl, methoxy, ethoxy, —CF₃, —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H, nitro, —CO₂R⁴⁻¹ or —SO₂R⁷⁻¹,

R²⁻¹ and R³⁻¹ furthermore together represent —O—CH₂—O—, —O—CH₂—CH₂—O—, —O—CF₂—O—, —O—CF₂—CF₂—O— if the two radicals are located ortho to one another,

R⁴⁻¹ represents methyl, ethyl, —CF₃, —CF₂CF₃ or —CF₂H,

R⁷⁻¹ represents fluorine, methyl, ethyl, —CF₃, —CF₂CF₃ or —CF₂H,

q represents 2.

The practice of the process according to the invention is illustrated by the examples below.

### PREPARATION EXAMPLES

### Example 1

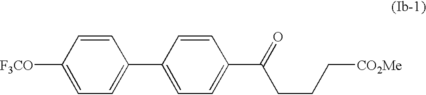

At -10° C., 450 ml of anhydrous hydrogen fluoride are initially charged in a stirred 11 autoclave made of V4A steel, 150 g (0.63 mol) of 4-trifluoromethoxybiphenyl are metered in and 95 g (0.63 mol) of methyl 4-oxo-4-chlorobutyrate are then added dropwise. The apparatus is then closed, and 80 g of boron trifluoride are added under pressure. The mixture is then stirred vigorously and, after warming to from +20° C. to +25° C., maintained at a pressure of 4 bar for 6 h.

For work-up, the mixture is heated to from +25° C. to +30° C., and the hydrogen fluoride/boron trifluoride is distilled off. The residue is discharged onto ice and dissolved with dichloromethane, and the organic phase is then, after washing with water, dried (azeotropic distillation). The crystalline residue is triturated with 150 ml of cold n-hexane and filtered off with suction.

This gives 117 g (53% of theory) of methyl 4-(4′-trifluoromethoxybiphenyl)-4-oxo-butyrate of melting point 125-127° C.

### Example 2

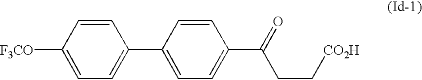

Analogously to Example 1, 119 g (0.5 mol) of 4-trifluoromethoxybiphenyl are reacted in a 0.51 autoclave with 50 g (0.5 mol) of succinic anhydride in the presence of 200 ml of anhydrous hydrogen fluoride and 50 g of boron trifluoride.

Work-up gives 135 g (80% of theory) of 4-(4′-trifluoromethoxybiphenyl)-4-oxo-butyric acid.

### Example 3

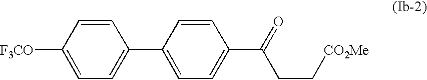

Analogously to Example 1, 136 g (0.61 mol) of 4-trifluoromethylbiphenyl are reacted with 92 g (0.61 mol) of methyl 4-oxo-4-chlorobutyrate in the presence of 500 ml of anhydrous hydrogen fluoride and 80 g of boron trifluoride.

After a 5-h-reaction at +25° C. and subsequent work-up, 154 g (48% of theory) of methyl 4-(4′-trifluoromethylbiphenyl)-4-oxobutyrate of melting point 140-141° C. are obtained.

### Example 4

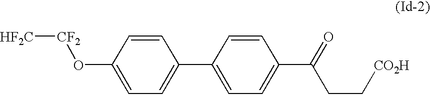

In a 11 autoclave made of stainless steel, 100 g (0.37 mol) of 1,1,2,2-tetrafluoro-ethoxybiphenyl, 200 ml of hydrogen fluoride, 39 g (0.39 mol) of succinic anhydride and 45 g of boron trifluoride are reacted as in Example 1 at from +20° C. to +25° C. for 7 h.

For work-up, 300 ml of dichloromethane are added to the mixture, which is then triturated. The organic phase is expelled via a riser pipe and added to 150 g of ice. Following trituration, the organic phase is separated off and the solvent is removed by azeotropic drying and azeotropic distillation. The crystalline residue is recrystallized from toluene.

This gives 107 g (78% of theory) of 4-oxo-4-[4′-(l,1,2,2-tetrafluoroethoxy)biphenyl-4-yl]butyric acid.

### Example 5

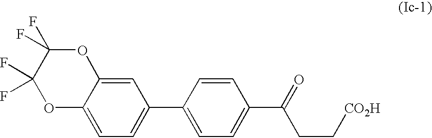

100 g (0.35 mol) of 2,2,3,3-tetrafluorobenzodioxenylbenzene, 250 ml of hydrogen fluoride, 40 g (0.40 mol) of succinic anhydride and 45 g of boron trifluoride are reacted analogously to Example 4.

Work-up gives 111 g (83% of theory) of 4-oxo-4-(3′,4′-tetrafluoroethylenedioxo-biphenyl-4-yl)butyric acid of melting point 165-168° C.

### Use Example

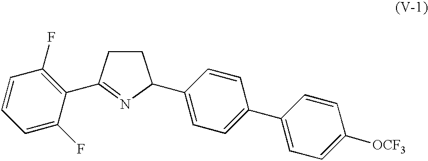

Step 1

Using a water separator, 4-oxo-4-[4′-(trifluoromethoxy)-1,1′-biphenyl-4-yl]butyric acid (Id-1) (11.00 g, 32.5 mmol), 2-(S)-2-amino-2-phenylethanol (4.46 g, 32.5 mmol), 4-toluenesulphonic acid (1.10 g, 5.8 mmol) and toluene (400 ml) are heated under reflux for 3.5 h. The reaction mixture is cooled, filtered and concentrated. The residue is triturated with diusopropyl ether and filtered off with suction.

This gives 5.56 g of 3-phenyl-7a-[4′-(trifluoromethoxy)-1,1′-biphenyl-4-yl]tetrahydropyrrolo[2,1-b][1,3]oxazol-5(6H)-one (VI-1) of melting point 104° C.

Step 2

Compound (VI-1) (3.81 g, 8.7 mmol) is initially charged in dichloromethane (75 ml), and triethylsilane (3.37 g, 29 mmol) and TiCl₄ (1 M solution in CH₂Cl₂, 19.1 ml, 19 mmol) are successively added dropwise at -78° C. The mixture is stirred at -78° C. for 2 h and then at room temperature overnight. The reaction mixture is cooled to 0° C. and saturated aqueous ammonium chloride solution (100 ml) is added dropwise. The organic phase is washed with water, dried over sodium sulphate, filtered and concentrated under reduced pressure. The crude product is reacted further without additional purification.

This gives 3.63 g of 1-[2-hydroxy-1-phenylethyl]-5-[4′-(trifluoromethoxy)-1,1′-biphenyl-4-yl]-2-pyrrolidinone (VII-1) [HPLC, log P (pH 2.3)=3.80].

Step 3

Compound (VII-1) (0.44 g, 1.0 mmol) is initially charged in THF (10 ml), and thionyl chloride (0.29 g, 2.42 mmol) is added dropwise. The reaction mixture is stirred for 1.5 h and then concentrated. The crude product is reacted further without additional purification.

This gives 0.38 g of 1-[2-chloro-1-phenylethyl]-5-[4′-(trifluoromethoxy)-1,1′-biphenyl-4-yl]-2-pyrrolidinone (VIII-1) [HPLC, log P (pH 2.3)=4.78].

Step 4

Compound (VIII-1) (0.50 g, 1.1 mmol) is initially charged in tert-BuOH (5 ml), and KO^{t}Bu (0.26 g, 2.4 mmnol) is added. The reaction mixture is stirred at 60° C. overnight, cooled and concentrated. The residue is taken up in ethyl acetate and washed successively with 1 M HCl and water. The organic phase is dried over magnesium sulphate, filtered and concentrated. The crude product is reacted further without additional purification.

This gives 0.34 g of 1-(1-phenylvinyl)-5-[4′-(trifluoromethoxy)-1,1′-biphenyl-4-yl]-2-pyrrolidinone (IX-1) [HPLC, log P (pH 2.3)=4.35]

Step 5

Compound (IX-1) (0.98 g, 2.3 mmol) is initially charged in THF (5 ml). 1 M HCl (5 ml) is added, the reaction mixture is stirred at 60° C. for 1 h and cooled to room temperature, and ethyl acetate (100 ml) is added. The organic phase is washed successively with saturated aqueous sodium bicarbonate solution and sodium chloride solution, dried over magnesium sulphate, filtered and concentrated. The crude product is reacted further without additional purification.

This gives 0.51 g of 5-[4′-(trifluoromethoxy)-1,1′-biphenyl-4-yl]-2-pyrrolidinone (X-1)[HPLC, log P (pH 2.3)=2.95].

Step 6

Compound (X-1) (0.51 g, 77.9% pure, about 1.23 mmol) is initially charged in dichloromethane (10 ml). t-Butoxycarbonyl anhydride (1.9 mmol, 0.56 g) and dimethylaminopyridine (0.02 g, 0.32 mmol) are added, and the reaction mixture is stirred at room temperature overnight. The mixture is diluted with dichloromethane (40 ml) and the organic phase is washed successively with 1 M HCl, saturated aqueous sodium bicarbonate solution and sodium chloride solution, dried over magnesium sulphate, filtered and concentrated under reduced pressure.

This gives 0.42 g (75% of theory) of tert-butyl 2-oxo-5-[4′-(trifluoromethoxy)-1,1′-biphenyl-4-yl]-1-pyrrolidinecarboxylate (XI-1) which is reacted further as a crude product without additional purification [HPLC, log P (pH 2.3)=4.32].

Step 7

At -78° C., 1,3-difluorobenzene (0.29 g, 2.55 mmol) is initially charged in THF (30 ml) under an atmosphere of argon. n-BuLi (1.6 M in hexane, 2.55 mmnol, 1.59 ml) and tetramethylethylenediamine (2.55 mmol, 0.38 ml) are successively added dropwise to this solution. The mixture is stirred at -78° C. for 20 min, and compound (XI-1) (1.70 mmol, 0.72 g) in TMF (2 ml) is then added dropwise at this temperature. Overnight, the reaction mixture is allowed to warm to room temperature, and is then poured into water (10 ml). The aqueous phase is extracted with ethyl acetate (100 ml) and the organic phase is washed with sodium chloride solution, dried over magnesium sulphate, filtered and concentrated under reduced pressure.

This gives 0.52 g of tert-butyl 4-(2,6-difluorophenyl)-4-oxo-1-[4′-(trifluoromethoxy)-1,1′-biphenyl-4-yl]butylcarbamate (XIII-1) which is reacted further as crude product without additional purification [HPLC, log P (pH 2.3)=5.18].

Step 8

At 0° C. compound (XIII-1) (0.10 g, 0.19 mmol) is initially charged in dichloromethane (5 ml). Trifluoroacetic acid (0.14 ml, 18.7 mmol) is added dropwise, and the reaction mixture is stirred at room temperature for 3 h and then concentrated to dryness. The residue is taken up in dichloromethane and adjusted to pH 12 using 2 M NaOH. The organic phase is washed with water, dried over magnesium sulphate, filtered and concentrated under reduced pressure.

This gives 0.09 g of (2R)-5-(2,6-difluorophenyl)-2-[4′-(trifluoromethoxy)-1,1′-biphenyl-4-yl]-3,4-dihydro-2H-pyrrole (V-1)[HPLC, log P (pH 2.3)=4.13].

The log P values given in the use examples above are determined in accordance with EEC Directive 79/831 Annex V.A8 by HPLC (High Performance Liquid Chromatography) on a reversed-phase column (C 18). Temperature: 43° C.

The determination is carried out in the acidic range at pH 2.3 using the mobile phases 0.1% aqueous phosphoric acid and acetonitrile; linear gradient from 10% acetonitrile to 90% acetonitrile.

Calibration is carried out using unbranched alkan-2-ones (with 3 to 16 carbon atoms) with known logP values (determination of the logP values by the retention times using linear interpolation between two successive alkanones).

The lambda max values were determined in the maxima of the chromatographic signals using the UV spectra from 200 nm to 400 nm.

1. Process for preparing ω-ketocarboxylic acid derivatives of the formula (I) 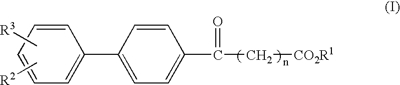
in which
R¹ represents hydrogen or straight-chain or branched alkyl,
R² represents halogen or in each case fluorine-substituted, in each case straight-chain or branched alkyl, alkoxy or alkylthio,
R³ represents hydrogen, halogen, in each case fluorine-substituted, in each case straight-chain or branched alkyl, alkoxy or alkylthio; represents in each case straight-chain or branched alkyl, alkoxy, alkylthio; cyano, nitro, —CO₂R⁴, —CONR⁵R⁶ or —SO₂R⁷,
R² and R³ furthermore together represent —O-(halo)alkyl-O—, if the two radicals are located ortho to one another,
R⁴ represents optionally fluorine-substituted straight-chain or branched alkyl,
R⁵ and R⁶ independently of one another represent hydrogen or straight-chain or branched alkyl,
R⁷ represents halogen or represents optionally fluorine-substituted straight-chain or branched alkyl,
n represents 1, 2, 3 or 4,
characterized in that compounds of the formula (II) 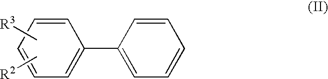
in which R² and R³ are as defined above are reacted either
a) with compounds of the formula (III) 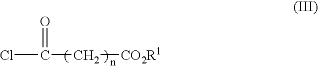
in which R¹ and n are as defined above or
b) with compounds of the formula (IV) 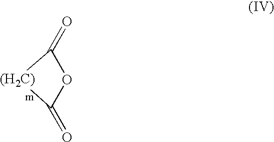
in which
m represents 2 or 3,
in anhydrous hydrogen fluoride, if appropriate in the presence of boron trifluoride.

2. Process according to claim 1, characterized in that compounds of the formula (II) 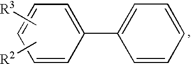
in which
R² represents fluorine, chlorine, bromine, iodine or represents in each case straight-chain or branched C₁-C₄-alkyl, C₁-C₄-alkoxy or C₁-C₄-alkylthio, each of which is substituted by 1 to 9 fluorine atoms,
R³ represents hydrogen, fluorine, chlorine, bromine, represents in each case straight-chain or branched C₁-C₄-alkyl, C₁-C₄-alkoxy, C₁-C₄-alkylthio, each of which is substituted by 1 to 9 fluorine atoms; represents in each case straight-chain or branched C₁-C₄-alkyl, C₁-C₄-alkoxy, C₁-C₄-alkylthio; cyano, nitro, —CO₂R⁴, —CONH₂ or —SO₂R⁷,
R² and R³ furthermore together represent —O—(C₁-C₄-alkyl)-O— if the two radicals are located ortho to one another, where the alkyl moiety may optionally be substituted by 1 to 8 fluorine atoms,
R⁴ represents straight-chain or branched C₁-C₄-alkyl which is optionally substituted by 1 to 9 fluorine atoms,
R⁷ represents fluorine, chlorine or represents straight-chain or branched C₁-C₄-alkyl which is optionally substituted by 1 to 9 fluorine atoms,
are used as starting materials.

3. Process according to claim 1, characterized in that compounds of the formula (II) 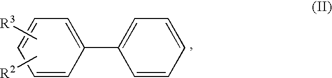
in which
R² represents fluorine, chlorine, bromine or represents methyl, ethyl, methoxy, ethoxy, methylthio or ethylthio, each of which is substituted by 1 to 5 fluorine atoms,
R³ represents hydrogen, fluorine, chlorine, represents methyl, ethyl, methoxy, ethoxy, methylthio or ethylthio, each of which is substituted by 1 to 5 fluorine atoms; represents methyl, ethyl, methoxy, ethoxy, nitro, —CO₂R⁴ or —SO₂R⁷,
R² and R³ furthermore together represent —O—CH₂—O— or —O—CH₂—CH₂—O— if the two radicals are located ortho to one another, where the methylene or ethylene moiety may optionally be substituted by 1 to 4 fluorine atoms,
R⁴ represents methyl or ethyl, optionally substituted by 1 to 5 fluorine atoms,
R⁷ represents fluorine, chlorine or represents methyl or ethyl, optionally substituted by 1 to 5 fluorine atoms,
are used as starting materials.

4. Process according to claim 1, characterized in that compounds of the formula (II) 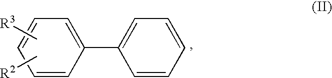
in which
R² represents fluorine, chlorine, bromine or represents —CF₃, —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H,
R³ represents hydrogen, fluorine, chlorine, methyl, ethyl, methoxy, ethoxy, —CF₃, —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H, nitro, —CO₂R⁴ or —SO₂R⁷,
R² and R³ furthermore together represent —O—CH₂—O—, —O—CH₂—CH₂—O—, —O—CF₂—O—, —O—CF₂—CF₂—O—, if the two radicals are located ortho to one another,
R⁴ represents methyl, ethyl, —CF₃, —CF₂CF₃ or —CF₂H,
R⁷ represents fluorine, methyl, ethyl, —CF₃, —CF₂CF₃ or —CF₂H,
are used as starting materials.

5. Process according to claim 1, characterized in that compounds of the formula (IIa) 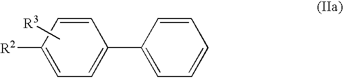
in which
R² and R³ are as defined in any of claims 1 to 4,
are used as starting materials.

6. Process according to claim 1, characterized in that compounds of the formula (IIb) 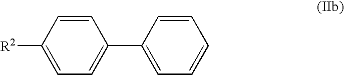
in which
R² is as defined in any of claims 1 to 4,
are used as starting materials.

7. Process according to claim 1, characterized in that compounds of the formula (III) 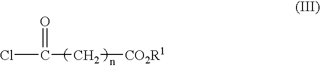
in which
R¹ represents hydrogen or straight-chain or branched C₁-C₄-alkyl,
n represents 2, 3 or 4,
are used as starting materials.

8. Process according to claim 1, characterized in that compounds of the formula (III) 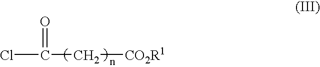
in which
R¹ represents hydrogen, methyl, ethyl, n-propyl or isopropyl,
n represents 2, 3 or 4,
are used as starting materials.

9. Process according to claim 1, characterized in that compounds of the formula (III) 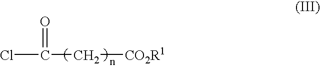
in which
R¹ represents hydrogen, methyl or ethyl,
n represents 2,
are used as starting materials.

10. Process according to claim 1, characterized in that the reaction is carried out at temperatures between -20° C. and +80° C.

11. Process according to claim 1, characterized in that the reaction is carried out at temperatures between -10° C. and +40° C.

12. Process according to claim 1, characterized in that the reaction is carried out at elevated pressure between 2 bar and 20 bar.

13. Process according to claim 1, characterized in that the reaction is carried out at elevated pressure between 3 bar and 10 bar.

14. Compounds of the formula (If) 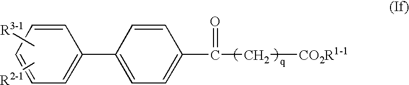
in which
a) R¹⁻¹ represents hydrogen or straight-chain or branched alkyl,
R²⁻¹ represents halogen or in each case fluorine-substituted, in each case straight-chain or branched alkyl, alkoxy or alkylthio,
R³⁻¹ represents hydrogen, halogen, represents in each case fluorine-substituted, in each case straight-chain or branched alkyl, alkoxy or alkylthio; represents in each case straight-chain or branched alkyl, alkoxy, alkylthio; cyano, nitro, —CO₂R⁴⁻¹, —CONR⁵⁻¹R⁶⁻¹ or —SO₂R⁷ ,
R²⁻¹ and R³⁻¹ furthermore together represent —O-(halo)alkyl-O— if the two radicals are located ortho to one another,
R⁴⁻¹ represents optionally fluorine-substituted straight-chain or branched alkyl,
R⁵⁻¹ and R⁶⁻¹ independently of one another represent hydrogen or straight-chain or branched alkyl,
R⁷⁻¹ represents halogen or represents optionally fluorine-substituted straight-chain or branched alkyl,
q represents 1, 3 or 4, or
b) R¹⁻¹ represents hydrogen or straight-chain or branched alkyl,
R²⁻¹ represents in each case fluorine-substituted, in each case straight-chain or branched C₂-C₆-alkyl, alkoxy or alkylthio,
R³⁻¹ represents hydrogen, halogen, represents in each case fluorine-substituted, in each case straight-chain or branched alkyl, alkoxy or alkylthio; represents in each case straight-chain or branched alkyl, alkoxy, alkylthio; cyano, nitro, —CO₂R⁴⁻¹, —CONR⁵⁻¹R⁶⁻¹ or —SO₂R⁷⁻¹,
R²⁻¹ and R³⁻¹ furthermore together represent —O-(halo)alkyl-O— if the two radicals are located ortho to one another,
R⁴⁻¹ represents optionally fluorine-substituted straight-chain or branched alkyl,
R⁵⁻¹ and R⁶⁻¹ independently of one another represent hydrogen or straight-chain or branched alkyl,
R⁷⁻¹ represents halogen or represents optionally fluorine-substituted straight-chain or branched alkyl,
q represents 2.

15. Compounds of the formula (If) according to claim 14 in which
a) R¹⁻¹ represents hydrogen or straight-chain or branched C₁-C₄-alkyl,
R²⁻¹ represents fluorine, chlorine, bromine, iodine or represents in each case straight-chain or branched C₁-C₄-alkyl, C₁-C₄-alkoxy or C₁-C₄-alkylthio, each of which is substituted by 1 to 9 fluorine atoms,
R³⁻¹ represents hydrogen, fluorine, chlorine, bromine, represents in each case straight-chain or branched C₁-C₄-alkyl, C₁-C₄-alkoxy, C₁-C₄-alkylthio, each of which is substituted by 1 to 9 fluorine atoms; represents in each case straight-chain or branched C₁-C₄-alkyl, C₁-C₄-alkoxy, C₁-C₄-alkylthio; cyano, nitro, —CO₂R⁴⁻¹, —CONH₂ or —SO₂R⁷⁻¹,
R²⁻¹ and R³⁻¹ furthermore together represent —O—(C₁-C₄-alkyl)-O— if the two radicals are located ortho to one another, where the alkyl moiety may optionally be substituted by 1 to 8 fluorine atoms,
R⁴⁻¹ represents straight-chain or branched C₁-C₄-alkyl which is optionally substituted by 1 to 9 fluorine atoms,
R⁷⁻¹ represents fluorine, chlorine or represents straight-chain or branched C₁-C₄-alkyl which is optionally substituted by 1 to 9 fluorine atoms,
q represents 3 or 4, or
b) R¹⁻¹ represents hydrogen or straight-chain or branched C₁-C₄-alkyly,
R²⁻¹ represents in each case straight-chain or branched C₂-C₄-alkyl, C₁-C₄-alkoxy or C₁-C₄-alkylthio, each of which is substituted by 1 to 9 fluorine atoms,
R³⁻¹ represents hydrogen, fluorine, chlorine, bromine, represents in each case straight-chain or branched C₁-C₄-alkyl, C₁-C₄-alkoxy, C₁-C₄-alkylthio, each of which is substituted by 1 to 9 fluorine atoms; represents in each case straight-chain or branched C₁-C₄-alkyl, C₁-C₄-alkoxy, C₁-C₄-alkylthio; cyano, nitro, —CO₂R⁴⁻¹, —CONH₂ or —SO₂R⁷⁻¹,
R²⁻¹ and R³⁻¹ together represent —O—(C₁-C₄-alkyl)-O— if the two radicals are located ortho to one. another, where the alkyl moiety may optionally be substituted by 1 to 8 fluorine atoms,
R⁴⁻¹ represents straight-chain or branched C₁-C₄-alkyl which is optionally substituted by 1 to 9 fluorine atoms,
R⁷⁻¹ represents fluorine, chlorine or represents straight-chain or branched C₁-C₄-alkyl which is optionally substituted by 1 to 9 fluorine atoms,
q represents 2.

16. Compounds of the formula (If) according to claim 14 in which
a) R¹⁻¹ represents hydrogen, methyl, ethyl, n-propyl or isopropyl,
R²⁻¹ represents fluorine, chlorine, bromine or represents methyl, ethyl, methoxy, ethoxy, methylthio or ethylthio, each of which is substituted by 1 to 5 fluorine atoms,
R³⁻¹ represents hydrogen, fluorine, chlorine, represents methyl, ethyl, methoxy, ethoxy, methylthio or ethylthio, each of which is substituted by 1 to 5 fluorine atoms; represents methyl, ethyl, methoxy, ethoxy, nitro, —CO₂R⁴⁻¹ or —SO₂R⁷⁻¹,
R²⁻¹ and R³⁻¹ furthermore together represent —O—CH₂—O— or —O—CH₂—CH₂—O— if the two radicals are located ortho to one another, where the methylene or ethylene moiety may optionally be substituted by 1 to 4 fluorine atoms,
R⁴⁻¹ represents methyl or ethyl, optionally substituted by 1 to 5 fluorine atoms,
R⁷⁻¹ represents fluorine, chlorine or represents methyl or ethyl, optionally substituted by 1 to 5 fluorine atoms,
q represents 3 or 4, or
b) R¹⁻¹ represents hydrogen, methyl, ethyl, n-propyl or isopropyl,
R²⁻¹ represents ethyl, methoxy, ethoxy, methylthio or ethylthio, each of which is substituted by 1 to 5 fluorine atoms,
R³⁻¹ represents hydrogen, fluorine, chlorine, represents methyl, ethyl, methoxy, ethoxy, methylthio or ethylthio, each of which is substituted by 1 to 5 fluorine atoms; represents methyl, ethyl, methoxy, ethoxy, nitro, —CO₂R⁴⁻¹ or —SO₂R⁷⁻¹,
R²⁻¹ and R³⁻¹ furthermore together represent —O—CH₂—O— or —O—CH₂—CH₂—O— if the two radicals are located ortho to one another, where the methylene or ethylene moiety may optionally be substituted by 1 to 4 fluorine atoms,
R⁴⁻¹ represents methyl or ethyl, optionally substituted by 1 to 5 fluorine atoms,
R⁷⁻¹ represents fluorine, chlorine or represents methyl or ethyl, optionally substituted by 1 to 5 fluorine atoms,
q represents 2.

17. Compounds of the formula (If) according to claim 14 in which
a) R¹⁻¹ represents hydrogen, methyl or ethyl,
R²⁻¹ represents fluorine, chlorine, bromine or represents —CF₃, —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H,
R³⁻¹ represents hydrogen, fluorine, chlorine, methyl, ethyl, methoxy, ethoxy, —CF₃, —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H, nitro, —CO₂R⁴⁻¹ or —SO₂R⁷⁻¹,
R²⁻¹ and R³⁻¹ furthermore together represent —O—CH₂—O—, —O—CH₂—CH₂—O—, —O—CF₂—O—, —O—CF₂—C₂—O— if the two radicals are located ortho to one another,
R⁴⁻¹ represents methyl, ethyl, —CF₃, —CF₂CF₃ or —CF₂H,
R⁷⁻¹ represents fluorine, methyl, ethyl, —CF₃, —CF₂CF₃ or —CF₂H,
q represents 3 or 4, or
b) R¹⁻¹ represents hydrogen, methyl or ethyl,
R²⁻¹ represents —CF₂CF₃, —CH₂CF₃, —CF₂CF₂H, —OCF₃, —OCF₂H, —OCF₂CF₃, —OCH₂CF₃, —OCF₂CF₂H, —SCF₃, —SCF₂H, —SCF₂CF₃, —SCH₂CF₃, —SCF₂CF₂H,
R³⁻¹ represents hydrogen, fluorine, chlorine, methyl, ethyl, methoxy, ethoxy, —CF₃, —CF₂CF₃, —CH₂CF₃, —CF₂CF₂H, —OCF₃, —OCF₂H, —OCF₂CF₃, —OCH₂CF₃, —OCF₂CF₂H, —SCF₃, —SC_{F}₂H, —SCF₂CF₃, —SCH₂CF₃, —SCF₂CF₂H, nitro, —CO₂R⁴⁻¹ or —SO₂R⁷⁻¹,
R²⁻¹ and R³⁻¹ furthermore together represent —O—CH₂—O—, —O—CH₂—CH₂—O—, —O—CF₂—O—, —O—CF₂—CF₂—O— if the two radicals are located ortho to one another,
R⁴⁻¹ represents methyl, ethyl, —CF₃, —CF₂CF₃ or —CF₂H,
R⁷⁻¹ represents fluorine, methyl, ethyl, —CF₃, —CF₂CF₃ or —CF₂H,
q represents 2.
